(19) United States
(12) Patent Application Publication
(10) Pub. No.: US 2022/0218225 A1
(43) Pub. Date: Jul. 14, 2022

(54) MEASURING DEVICE AND MEASURING METHOD

(71) Applicant: TERUMO KABUSHIKI KAISHA, Tokyo (JP)

(72) Inventors: Shinichiro Suda, Kanagawa (JP); Kei Honda, Kanagawa (JP)

(21) Appl. No.: 17/707,242

(22) Filed: Mar. 29, 2022

Related U.S. Application Data
(63) Continuation of application No. PCT/JP2020/037175, filed on Sep. 30, 2020.

(30) Foreign Application Priority Data
Sep. 30, 2019 (JP) 2019-179213

(51) Int. Cl. A61B 5/0507; A61B 5/024; A61B 5/00

(52) U.S. Cl. CPC A61B 5/0507; A61B 5/0026; A61B 5/725; A61B 5/024

(57) ABSTRACT

A measuring device and a measuring method according to embodiments of the present disclosure can measure an index related to a heart with high accuracy. The present disclosure provides a measuring device configured to measure an index related to a heart of a living body. The measuring device includes: a transmitting unit and a receiving unit that can transmit microwaves at a plurality of different locations of the living body and measure the transmitted microwaves; a detecting unit that can acquire waveform parameters of the microwaves measured at the plurality of locations and compare the waveform parameters; and a detecting unit that can position the transmitting unit and the receiving unit that measure the microwave used to calculate the index among the microwaves measured at the plurality of locations based on a comparison result obtained by the detecting unit.

### CROSS-REFERENCE TO RELATED APPLICATIONS

The present application is a continuation of and claims benefit to PCT Application No. PCT/JP2020/037175 filed on Sep. 30, 2020, entitled “MEASUREMENT DEVICE AND MEASUREMENT METHOD” which claims priority to Japanese Patent Application No. 2019-179213 filed on Sep. 30, 2019. The entire disclosures of the applications listed above are hereby incorporated herein by reference, in their entirety, for all that they teach and for all purposes.

### BACKGROUND

The present disclosure relates to a measuring device and a measuring method.

In the related art relating to the detection of a cardiac output, for example, a device described in International Publication No. WO2018/194093 includes a transmitting antenna, a receiving antenna, and an estimating unit. In the above device, the transmitting antenna transmits a microwave or the like to a chest of a patient, the receiving antenna receives the microwave or the like transmitted from the transmitting antenna, and the estimating unit detects the cardiac output of a measurement target based on a phase or an amplitude intensity of the microwave received by the receiving antenna.

However, depending on a position of the transmitting antenna that transmits the microwave with respect to a living body, a position of the receiving antenna, or a positional relationship between the transmitting antenna and the receiving antenna, a received wave obtained by the receiving antenna may not be suitable for the measurement of the cardiac output. When the transmitting and receiving antennas are used in a state unsuitable for measuring the cardiac output to be obtained, the accuracy of measuring the cardiac output is reduced. Therefore, there is a demand for a technique capable of measuring an index related to a heart, such as the cardiac output, with high accuracy.

Furthermore, when a change in the cardiac output for each measurement is used for medical care, if there is no technique for setting a position of the transmitting antenna or the receiving antenna at locations suitable for the measurement, there is a problem that it is not possible to determine whether the obtained change in the cardiac output is due to a change in an arrangement position of the antenna or a change in a patient state.

### BRIEF SUMMARY

Accordingly, at least one object of the present disclosure is to provide a measuring device and a measuring method that can measure an index related to a heart with high accuracy.

In order to achieve the above object, the present disclosure provides, for example, a measuring device configured to measure an index related to a heart of a living body. The measuring device includes: a measuring unit configured to transmit microwaves at a plurality of different locations of the living body and measure the transmitted microwaves; a comparing unit configured to acquire waveform parameters of the microwaves measured at the plurality of locations and compare the waveform parameters; and a positioning unit configured to position the measuring unit that measures the microwave used to calculate the index among the microwaves measured at the plurality of locations based on a comparison result obtained by the comparing unit.

The present disclosure provides a measuring method for measuring an index related to a heart of a living body. The measuring method includes: transmitting microwaves at a plurality of different locations of the living body and measuring the transmitted microwaves; acquiring waveform parameters of the microwaves measured at the plurality of locations and comparing the waveform parameters; and positioning a measurement location of the microwave used to calculate the index among the microwaves measured at the plurality of locations based on a comparison result of the waveform parameters.

According to the measuring device and the measuring method in the present disclosure, it is possible to measure an index related to a heart with relatively high accuracy.

According to the measuring device and the measuring method in the present disclosure, it is possible to measure a cardiac output or the like, which is an index related to the heart, regardless of the skill and motion of a medical worker. As a result, it is possible to accurately grasp the transition of a heart state of a patient day by day or on a certain day.

### BRIEF DESCRIPTION OF THE DRAWINGS

FIG. 1 is a schematic perspective view illustrating a measuring device according to embodiments of the present disclosure;

FIG. 2 is a side view illustrating the measuring device of FIG. 1 according to embodiments of the present disclosure;

FIG. 3 is a block diagram illustrating a configuration of the measuring device of FIG. 1 according to embodiments of the present disclosure;

FIG. 4 is a diagram illustrating a case in which a transmitting unit and a receiving unit are positioned with respect to a heart of a patient or subject according to embodiments of the present disclosure;

FIG. 5 is a flowchart illustrating a case in which a cardiac output of the patient is measured using the measuring device of FIG. 1 according to embodiments of the present disclosure;

FIG. 6 is a diagram illustrating a case in which the receiving unit of the measuring device of FIG. 1 is displaced with respect to the heart of the patient according to embodiments of the present disclosure;

FIG. 7 is a schematic waveform diagram illustrating the cardiac output of the patient measured at a position of the receiving unit of FIG. 6 according to embodiments of the present disclosure;

FIG. 8 is a diagram illustrating a case in which a receiving unit of the measuring device is aligned with the heart of the patient according to embodiments of the present disclosure;

FIG. 9 is a schematic waveform diagram illustrating the cardiac output of the patient as measured at a position of the receiving unit of FIG. 8 according to embodiments of the present disclosure;

FIG. 10 is a waveform diagram illustrating a microwave used for calculating a cardiac output, which is an example of an index related to a heart, that can be selected in a measuring device according to embodiments of the present disclosure;

FIG. 11 is a waveform diagram illustrating a microwave used for calculating the cardiac output, which is the example of the index related to the heart, that can be selected in the measuring device according to embodiments of the present disclosure; and

FIG. 12 is a diagram illustrating an arrangement (positional relationship) between a transmitting unit and a receiving unit in a measuring device according to embodiments of the present disclosure.

### DETAILED DESCRIPTION

Hereinafter, embodiments of the present disclosure will be described with reference to accompanying drawings. In the descriptions of the drawings, the same elements are denoted by the same reference numerals, and redundant descriptions thereof will be omitted. Sizes and ratios of the members in the drawings may be exaggerated for convenience of description, and may be different from actual sizes and ratios.

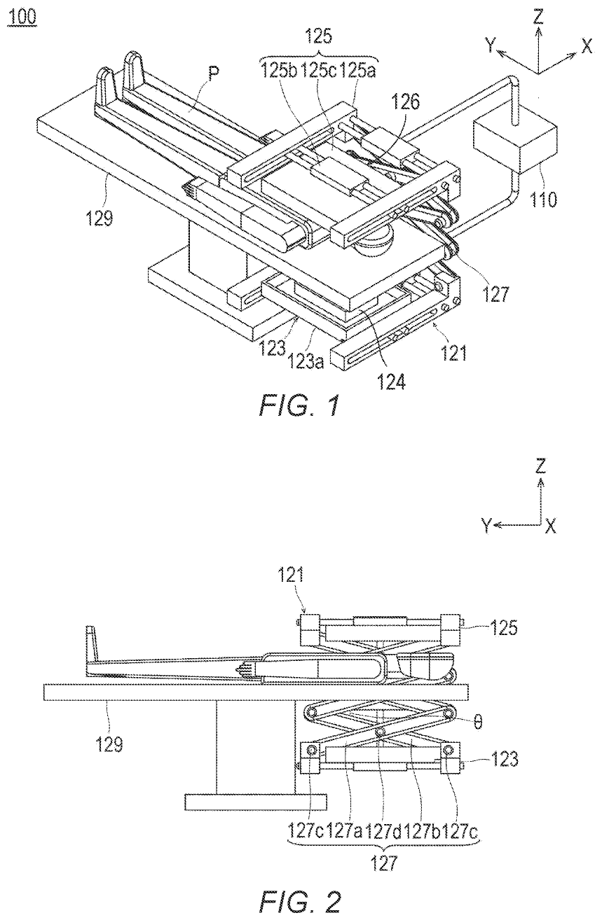

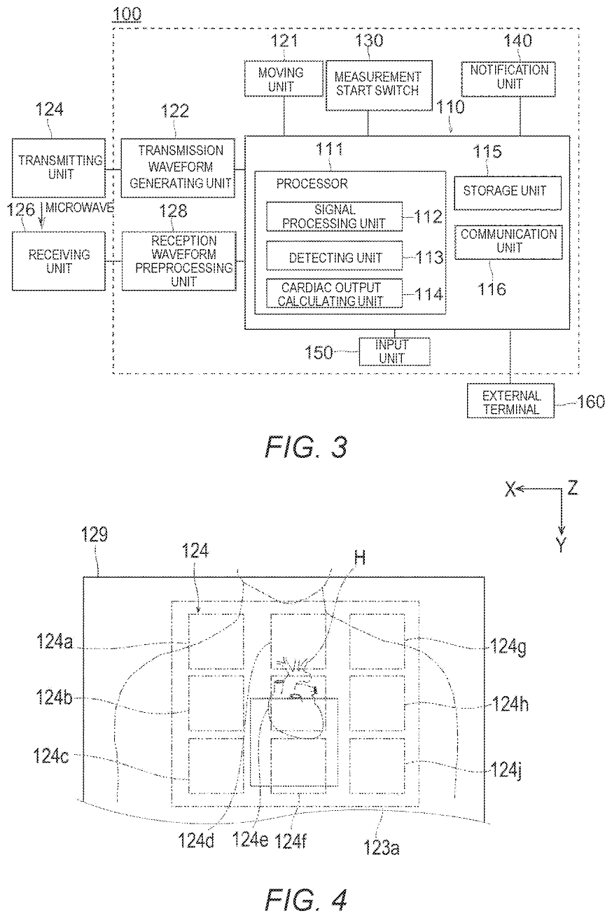

FIGS. 1 and 2 are a schematic perspective view and a side view illustrating a measuring device 100 according to at least one embodiment of the present disclosure, respectively, and FIG. 3 is a block diagram illustrating aspects of the measuring device 100 of FIG. 1. FIG. 4 is a diagram illustrating a transmitting unit 124 and a receiving unit 126 positioned with respect to a heart H of a patient.

The measuring device 100 illustrated in FIGS. 1 and 3 can measure an index related to the heart H of a living body such as a cardiac output in the living body of a patient P or subject in, for example, an examination of heart failure, an elapse observation after surgery of the heart H, and verification of a medication effect, a side effect, and the like of a heart disease.

As illustrated in FIGS. 1 and 3, the measuring device 100 includes the transmitting unit 124, the receiving unit 126, a transmission waveform generating unit 122, and a reception waveform preprocessing unit 128. The measuring device 100 includes a moving unit 121, a control unit 110, a measurement start switch 130, a notification unit 140, and an input unit 150. The measuring device 100 can communicate with an external terminal 160.

In some embodiments, the processor 111 may correspond to one or more computer processing devices. For example, the processor 111 and/or one or more components thereof (e.g., the signal processing unit 112, the detecting unit 113, the cardiac output calculating unit 114, etc.) may be provided as silicon, an Application-Specific Integrated Circuit (“ASIC”), as a Field Programmable Gate Array (“FPGA”), any other type of Integrated Circuit (“IC”) chip, a collection of IC chips, and/or the like. In some embodiments, the processor 111 and/or one or more components thereof may be provided as a Central Processing Unit (“CPU”), a microprocessor, or a plurality of microprocessors that are configured to execute the instructions sets. In some embodiments, the components of the processor 111 e.g., the signal processing unit 112, the detecting unit 113, the cardiac output calculating unit 114, etc.) may be embodied as a virtual processor(s) executing on one or more physical processors. The execution of a virtual processor may be distributed over a number of physical processors or one physical processor may execute one or more virtual processors. Virtual processors are presented to a process as a physical processor for the execution of the process while the specific underlying physical processor(s) may be dynamically allocated before or during the execution of the virtual processor wherein the instruction stack and pointer, register contents, and/or other values maintained by the virtual processor for the execution of the process are transferred to another physical processor(s). As a benefit, the physical processors may be added, removed, or reallocated without affecting the virtual processors execution of the processes. Additionally or alternatively, the physical processor(s) may execute a virtual processor to provide an alternative instruction sets as compared to the instruction set of the virtual processor (e.g., an “emulator”). As a benefit, a process compiled to run a processor having a first instruction set (e.g., Virtual Address Extension (“VAX”)) may be executed by a processor executing a second instruction set (e.g., Intel® 9xx chipset code) by executing a virtual processor having the first instruction set (e.g., VAX emulator).

The transmission waveform generating unit 122, the transmitting unit 124, the receiving unit 126, the reception waveform preprocessing unit 128, the moving unit 121, and a signal processing unit 112 of the control unit 110 transmit microwaves at a plurality of different locations of the living body, and measure the transmitted microwaves. The transmission waveform generating unit 122, the transmitting unit 124, the receiving unit 126, the reception waveform preprocessing unit 128, the moving unit 121, and the signal processing unit 112 of the control unit 110 correspond to a measuring unit in at least one embodiment.

The transmitting unit 124, or transmitter, is electrically connected to the transmission waveform generating unit 122, and can transmit microwaves to the living body of the patient P. As illustrated in FIG. 4, the transmitting unit 124 is disposed in a table 123a of a first placing portion 123 of the moving unit 121 to be described later. The transmitting unit 124 may be disposed on a back side of a human body of the patient P in a state in which the patient P lies supine on a bed 129.

The transmitting unit 124 includes a plurality of transmitting locations of microwaves, and in the present embodiment, as illustrated in FIG. 4, nine transmitting antennas 124a to 124i are provided as the plurality of transmitting locations of microwaves. For the transmitting antennas 124a to 124i, three transmitting antennas are arranged in a first direction X along a first side of the bed 129 in the table 123a of the first placing portion 123, and three rows of combinations of three transmitting antennas arranged in the first direction X are arranged in a second direction Y along a second side intersecting the first side of the bed 129. However, a specific mode of the transmitting antennas is not limited to the above as long as the microwaves can be transmitted from the plurality of different locations to the bed 129 on which the patient P can be placed. The sizes, ratios, shapes, and hardness of the members or the number of antennas are exaggerated for convenience of description, and may be different from the actual sizes and ratios. The transmitting unit 124 can switch between a transmission state and a non-transmission state (e.g., switch between being on and being off) of microwaves according to a certain order when the plurality of transmitting locations of microwaves are provided as the transmitting antennas 124a to 124i.

In the receiving unit 126, or receiver, one receiving antenna is disposed at any position facing the plurality of transmitting antennas 124a to 124i corresponding to the plurality of transmitting locations in a second placing portion 125. The receiving unit 126 is movable by the moving unit 121, so that the receiving unit 126 is capable of receiving microwaves according to any position of the transmitting antennas 124a to 124i constituting the transmitting unit 124. The receiving unit 126 is disposed in a table 125c of the second placing portion 125 to be described later. The receiving unit 126 is disposed on a front side of the human body of the patient P while the patient P lies supine on the bed 129.

The transmitting unit 124 and the receiving unit 126 can be implemented by a dipole-type linear antenna or the like. However, the formats of the transmitting unit 124 and the receiving unit 126 are not particularly limited as long as microwaves can be transmitted and received. The transmitting unit 124 and the receiving unit 126 may be a linear antenna of a minute loop type or a helical type, or may be a planar antenna of a patch type or an inverted F type.

The transmission waveform generating unit 122 is implemented by a microwave generator. A frequency of a microwave to be generated is not particularly limited as long as the microwave can pass through the heart H of the human body, and may be, for example, a frequency of approximately 1 Gigahertz (GHz) or a frequency of approximately 400 Megahertz (MHz). The power of the microwave generated is not particularly limited as long as sufficient power can be detected by the receiving unit 126, and may be, for example, several milliwatts (mW) to several tens of mW (e.g., between 2 mW and 30 mW). It is desirable to set the frequency of the microwave to be generated at which the waveform for obtaining a cardiac output is obtained most clearly, and the microwave to be generated may be a continuous wave, a pulse wave, or an electromagnetic wave that is subjected to phase modulation or frequency modulation.

The reception waveform preprocessing unit 128 executes preprocessing such as analog-to-digital (A/D) conversion such that the control unit 110 to be described later can process the microwaves received from the receiving unit 126. The reception waveform preprocessing unit 128 can be implemented by, for example, an A/D converter or the like.

The moving unit 121 can move (e.g., change) the relative positions of the transmitting unit 124 and the receiving unit 126 with respect to the patient P. As illustrated in FIG. 1 and the like, the moving unit 121 includes the first placing portion 123, the second placing portion 125, a vertical unit 127, and the bed 129.

The first placing portion 123 is disposed below the bed 129 on which the patient P is placed. The first placing portion 123 includes the table 123a capable of placing a plurality of transmitting antennas 124a to 124i which are the above-mentioned transmitting locations of microwaves and constitute the transmitting unit 124.

As illustrated in FIG. 4, the table 123a has a shape that is a planar pedestal larger than the heart H of the patient P in a plan view from a third direction Z in which microwaves are transmitted. In some embodiments, the first placing portion 123 may be fixed with respect to the bed 129 in the first direction X and the second direction Y. In this fixed arrangement, the first placing portion 123 may not move.

In some embodiments, the second placing portion 125 is disposed above the bed 129, on which the patient P is placed, on an opposite side of the first placing portion 123. As illustrated in FIG. 1, the second placing portion 125 includes a rail 125a, a drive unit 125b, and the table 125c. In one example, the second placing portion may correspond to a cartesian coordinate robot and/or a linear actuator having rails and moving carriage. In this example, the receiving unit 126 may be attached to the moving carriage. In any event, the receiving unit 126 is movable in the first direction X and the second direction Y, that is, is linearly movable by the second placing portion 125.

In the present embodiment, the rail 125a is implemented by a pair of long members extending in the first direction X, and can move the drive unit 125b and the table 125c back and forth in the first direction X by a motor or the like (not illustrated). In some embodiments, the first direction X corresponds to a lateral direction (e.g., a direction of the first side) of the bed 129.

The drive unit 125b can move the table 125c in the second direction Y intersecting the first direction X. The drive unit 123b can be implemented by a motor, a ball screw, and/or the like (not illustrated). In some embodiments, the second direction Y corresponds to a longitudinal direction (e.g., a direction of the second side intersecting the first side) of the bed 129.

The table 125c is attached to the drive unit 125b, and is movable in the first direction X and the second direction Y together with the drive unit 125b. The receiving unit 126 is mounted on the table 125c.

The vertical unit 127 causes the first placing portion 123 and the second placing portion 125 to be relatively close to and separated from each other. As illustrated in FIG. 2, the vertical unit 127 includes first link members 127a, second link members 127b, and pins 127c and 127d. The transmitting unit 124 and the receiving unit 126 are movable in the third direction Z, that is, linearly movable by the vertical unit 127, and can be relatively close to and separated from each other. In some examples, the vertical unit 127 may correspond to a scissor lift, scissor jack, and/or scissor-lift mechanism.

As illustrated in FIG. 2, a plurality of the first link members 127a and a plurality of the second link members 127b are provided in the third direction Z. The plurality of first link members 127a face an obliquely upper right direction in the third direction Z in FIG. 2. The plurality of second link members 127b face a direction different from that of the first link members 127a in the third direction Z in FIG. 2, that is, an upper left direction.

The first link members 127a and the second link members 127b are rotatably connected by the pins 127c disposed at an end portion in the second direction Y. The pin 127d rotatably connects the first link member 127a and the second link member 127b in a middle of the pins 127c adjacent in the third direction Z in addition to the pins 127c.

When an angle θ (see FIG. 2) formed on a center side of the first link member 127a and the second link member 127b that are connected by the pins 127c and 127d is close to 0 degrees, the first placing portion 123 and the second placing portion 125 are relatively brought close to each other. On the other hand, when the angle θ formed by the first link member 127a and the second link member 127b is close to 180 degrees, the first placing portion 123 and the second placing portion 125 are relatively separated from each other.

The bed 129 is disposed in a direction orthogonal to a ground from a pedestal mounted on the ground, extends substantially along the ground, and has a flat surface capable of supporting the patient P (e.g., a child, an adult, etc.). The bed 129 includes a rectangular table having a short side along the first direction X and a long side along the second direction Y in the plan view from the third direction Z in which microwaves are transmitted. In some embodiments, the table of the bed 129 is formed in a size according to a body size of an adult, and a slide mechanism may be mounted so that the size of the table can be changed in a plurality of stages according to a body size of the patient P.

As illustrated in FIG. 3, the control unit 110 includes a processor 111 such as a CPU, a storage unit 115, and a communication unit 116. The processor 111, the storage unit 115, and the communication unit 116 are connected to one another by a bus (not illustrated). The storage unit 115 may comprise a memory, or memory storage device, which may correspond to any type of non-transitory computer-readable medium. In some embodiments, the memory may comprise volatile or non-volatile memory and a controller for the same. Non-limiting examples of the memory that may be utilized in the control unit 110 may include Random Access Memory (“RAM”), Read Only Memory (“ROM”), buffer memory, flash memory, solid-state memory, or variants thereof. Any of these memory types may be considered non-transitory computer memory devices even though the data stored thereby can be changed one or more times. The memory may be used to store information about communications, identifications, conditional requirements, times, historical data, and/or the like. In some embodiments, the memory may be configured to store rules and/or the instruction sets in addition to temporarily storing data for the processor 111 to execute various types of routines or functions (e.g., signal processing, cardiac output calculating, etc.). Although not depicted, the memory may include instructions that enable the processor 111 to store data into a memory storage device and retrieve information from the memory storage device. In some embodiments, the memory storage device or the data stored therein may be stored internal to the measuring device 100.

In some embodiments, as illustrated in FIG. 3, the processor 111 functions as the signal processing unit 112, a detecting unit 113 (corresponding to a comparing unit and a positioning unit), and a cardiac output calculating unit 114.

The signal processing unit 112 removes unnecessary components such as noise in the waveforms acquired from the reception waveform preprocessing unit 128. The signal processing unit 112 is not particularly limited, and can perform a known filtering process by a band-pass filter or the like on the waveforms acquired from the reception waveform preprocessing unit 128, for example.

The detecting unit 113 acquires and compares the waveform parameters of the waveforms (e.g., waveforms processed by the signal processing unit 112) of the microwaves received by the receiving unit 126 at a plurality of locations relatively different from one another with respect to the bed 129. The detecting unit 113 detects a position at which a waveform parameter is maximum among the microwaves received at the plurality of locations based on a comparison result of the above-described waveforms. Accordingly, the transmitting unit 124 and the receiving unit 126 are positioned in which the microwave used for calculation of the index related to the heart H such as the cardiac output is measured.

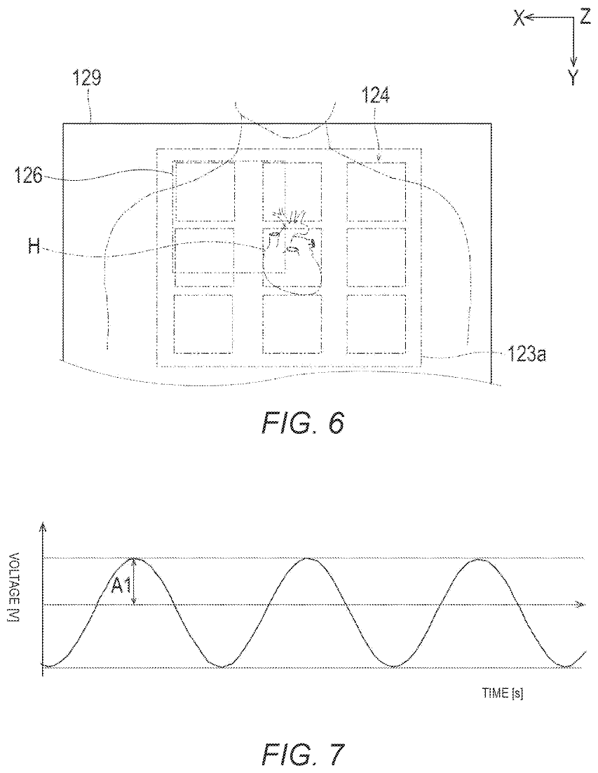

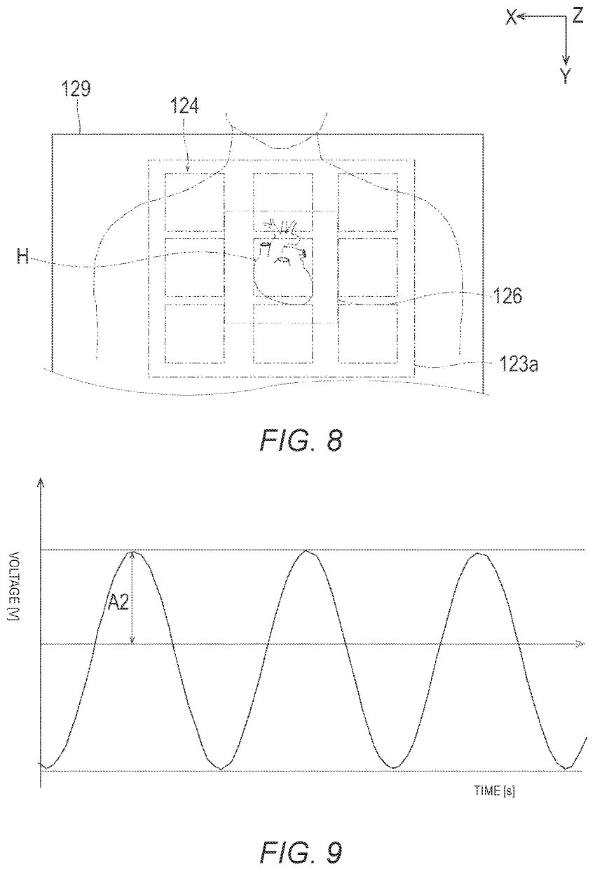

FIG. 6 is a diagram illustrating a case in which the receiving unit 126 of the measuring device 100 is displaced with respect to the heart H of the patient P, and FIG. 7 is a waveform diagram illustrating a case in which the cardiac output of the patient P is measured at a position of the receiving unit according to FIG. 6. FIG. 8 is a diagram illustrating a case in which the receiving unit 126 of the measuring device 100 is provided according to the heart H of the patient P as compared with the case in FIG. 6, and FIG. 9 is a waveform diagram illustrating a case in which the cardiac output of the patient P is measured at a position of the receiving unit 126 according to FIG. 8.

When the received microwaves are analyzed to calculate the index related to the heart H, such as the cardiac output, as a signal level of the fluctuation associated with a beating of a heart is higher, a periodic fluctuation due to the beating of the heart appears more clearly, and an amplitude of the fluctuation becomes larger. In contrast, as the signal level of the fluctuation due to the beating of the heart is lower, the periodic fluctuation due to the beating of the heart is buried in noise, and the amplitude of the fluctuation decreases.

That is, as illustrated in FIG. 8, as the position of the receiving unit 126 is closer to the heart H, the periodic fluctuation due to the beating of the heart H appears more clearly, and an amplitude A2 of the fluctuation of the voltage becomes larger as illustrated in FIG. 9. On the contrary, as illustrated in FIG. 6, as the position of the receiving unit 126 moves away from the heart H, the periodic fluctuation due to the beating of the heart H is buried in noise, and the amplitude A1 of the fluctuation of the voltage decreases as illustrated in FIG. 7.

With the above in mind, the detecting unit 113 compares the waveform parameters of the microwaves acquired at the plurality of different locations by the receiving unit 126, and positions the transmitting unit 124 and the receiving unit 126 that measure a microwave having the maximum waveform parameter. Accordingly, it is possible to measure the index related to the heart, such as the cardiac output, at a position near the heart where the periodic fluctuation of the heart is likely to appear. In some embodiments, the waveform parameter is an amplitude of the microwave received by the receiving unit 126, but the waveform parameter is not limited to an amplitude intensity as long as the signal intensity of a fluctuation component due to the beating of the heart can be evaluated. For example, a waveform area at one wavelength may be used instead of the amplitude intensity of the waveform.

In some embodiments, the cardiac output calculating unit 114 calculates an index related to the heart H of the patient P such as a cardiac output at a position where the waveform parameter is the largest among the waveforms compared by the detecting unit 113.

The storage unit 115 stores the waveform parameters of the received microwave at each position and at each time point. The storage unit 115 stores a position of the transmitting unit 124 at which the waveform parameter is maximum at a plurality of time points at a time interval, such as a first time point and a second time point. Specifically, the storage unit 115 stores a transmitting antenna number that specifies a transmitting antenna having the maximum waveform parameter, a coordinate of the transmitting antenna, and the like.

As will be described later, the storage unit 115 can store a program or the like for transmitting microwaves from the plurality of different locations using the transmitting unit 124 to the patient P lying supine on the bed 129. The program includes contents specifying that microwaves are transmitted in a certain order at the transmitting antennas 124a to 124i. The storage unit 115 can be implemented by a ROM, RAM, or the like.

The communication unit 116 enables transmission and reception of data to and from a device different from the measuring device 100, such as the external terminal 160. The communication unit 116 enables wired or wireless communication with the external terminal 160. The communication unit 116 can be implemented by, for example, a port (e.g., an interface) of a wired cable such as a network card or a universal serial bus (USB).

The measurement start switch 130 can instruct the start of the measurement by a user (e.g., a medical worker such as a doctor or a nurse). A specific mode of the measurement start switch 130 is not particularly limited as long as the on and off switching can be performed, and examples thereof include a toggle type switch and a button type switch.

The notification unit 140 notifies which index related to the heart H of the patient P is being acquired by the control unit 110 (e.g., by the various units of the control unit 110, etc.), such as the cardiac output.

The notification unit 140 gives a notification of a measurement value of the index related to the heart, such as the cardiac output. A specific mode of the notification unit 140 is not particularly limited as long as the notification unit 140 can notify the user of the measurement value of the index related to the heart, and the notification unit 140 can provide, for example, a notification audibly (e.g., via a speaker, etc.) and/or visibly by displaying a measurement result on a display (e.g., a display device, etc.). The notification unit 140 may notify that the patient P is not present on the bed 129 by a buzzer or the like. The notification unit 140 may give a notification of the index related to the heart such as the cardiac output of the patient P or a comparison result related to a sensor position by voice, light, or the like. In some examples, the notification unit 140 may comprise at least one graphical user interface, speaker, light, display device, and/or touchscreen.

By the input unit 150, the user such as the medical worker can input information related to the patient P to the measuring device 100. The input unit 150 can be implemented by any one or more of a push button, a keyboard, a pointing device such as a mouse, a graphical user interface, and the like, or a combination thereof in whole or in part. In some embodiments, the input unit 150 may be an element of the measuring device 100, and, additionally, may be disposed externally from the measuring device 100. In one example, the input unit 150 may be a part of the notification unit 140 when the notification unit 140 comprises a graphical user interface.

The external terminal 160 can communicate data of an index related to the heart H with the measuring device 100 through the communication unit 116. The external terminal 160 can be implemented by a known tablet (e.g., a terminal a user may interact with using touch, typing, voice commands, etc., and/or combinations thereof), a personal computer, or the like.

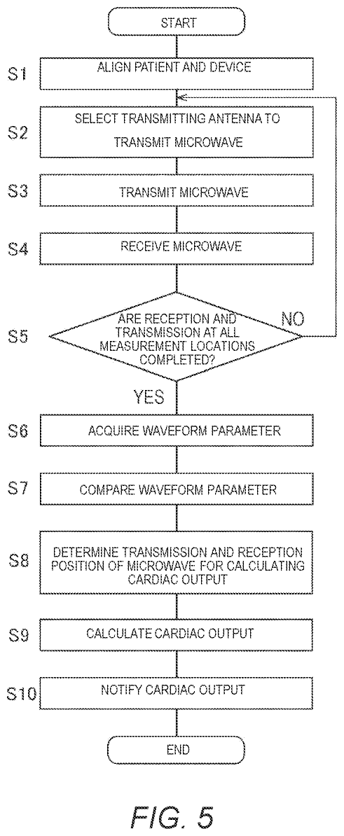

Turning next to FIG. 5, a flowchart illustrating a measuring method is shown according to embodiments of the present disclosure. To give a general description of the measuring method, the positioning (S1) of a patient and a device, the selection (S2) of a transmitting antenna that transmits microwaves, the transmission and reception (S3, S4) of the microwaves, and the acquisition and comparison (S6, S7) of waveform parameters are executed. In the measuring method described above, the determination (S8) of the microwave transmission and reception position for calculating a cardiac output, the calculation (S9) of the cardiac output, and the notification (S10) of an index are executed.

The measuring device 100 receives an input from the input unit 150 by the user, and acquires information related to the patient P such as an ID of the patient P. At this time, in order to calculate a value of the cardiac output or the like as an index related to a heart state of the patient P, information such as a body weight, a height, a chest thickness, a chest circumference, and a chest width may be input (e.g., via the input unit 150, etc.). Next, the patient P receives an instruction from the user of the measuring device 100, such as the medical worker, and lies supine on the bed 129. Accordingly, as illustrated in FIG. 1 and the like, the transmitting unit 124 and the receiving unit 126 are roughly aligned with a position near the heart of the patient P in a state in which the measuring device 100 is viewed in the plan view from the third direction Z (S1).

A distance between an antenna and a body surface of the patient P may be automatically acquired by an infrared sensor or the like near the transmitting antenna or the receiving antenna, and/or a position and tilt of the antenna may be acquired as data by an acceleration sensor or the like.

Next, when the measurement start switch 130 is operated by the user, a microwave transmission and reception program stored in the storage unit 115 is read by the processor 111. The processor 111 selects a transmitting antenna that transmits a microwave according to the read program (S2).

The processor 111 causes the microwave to be transmitted from the transmitting antenna selected according to the program (S3). The receiving unit 126 is moved by the second placing portion 125 according to a position of the transmitting antenna that transmits the microwave in response to an instruction of the processor 111, and receives the microwave transmitted through the living body of the patient P (S4).

In some embodiments, as an example, transmitting antennas that transmit microwaves are selected in alphabetical order of the transmitting antennas 124a to 124i (S2). Until the microwaves are transmitted and received at all the measurement locations (S5: NO), the transmission (S3) of the microwaves from the transmitting unit 124 and the reception (S4) of the microwaves executed by the receiving unit 126 are repeated.

In some embodiments, the microwaves are transmitted from all the transmitting antennas 124a to 124i, and steps S2, S3, and S4 in a flowchart illustrated in FIG. 6 are repeated until the receiving unit 126 receives the microwaves at all the measurement locations according to the position of the transmitting unit 124.

In some embodiments, it is set in advance how many times (e.g., a predetermined number of times, etc.) the position of the receiving unit 126 is changed for each of the transmitting antennas 124a to 124i to complete the transmission and reception at all the measurement locations. For example, a position at which a central axis of the antenna in a Z-axis direction is aligned with respect to one transmitting antenna is set as an initial placing position of the receiving unit 126, and it is set that microwaves are transmitted and received at locations separated from that position obliquely in an upper-lower direction and a left-right direction by 0.5 cm and 1.0 cm in parallel with an XY plane. Accordingly, the position of the receiving unit 126 is changed at 17 locations for one transmitting antenna to perform the measurement. Steps S2, S3, and S4 in the flowchart illustrated in FIG. 6 are repeated 17 times for the one transmitting antenna. By performing this operation on all of the transmitting antennas 124a to 124i, steps S2, S3, and S4 in the flowchart illustrated in FIG. 6 are repeated 153 times.

When the microwaves are transmitted and received at all the measurement locations (S5: YES), the reception waveform preprocessing unit 128 converts the microwaves received by the receiving unit 126 from analog to digital signals. The signal processing unit 112 executes numerical analysis and filtering of unnecessary information on the signal (data) obtained by converting the microwaves by the reception waveform preprocessing unit 128, and acquires an amplitude, which is a waveform parameter of the microwave, for each of a plurality of locations at which the microwave is transmitted and received (S6).

The detecting unit 113 compares the waveform parameters acquired by the signal processing unit 112 in the waveforms received by the receiving unit 126 at the plurality of different locations of the patient P (S7). Then, a combination of placing positions of the transmitting antenna and the receiving unit 126 having the maximum amplitude, which is a waveform parameter, is selected (S8). Accordingly, among the microwaves measured at the plurality of locations, the measurement locations of the microwaves used for calculating the cardiac output are positioned.

When the detecting unit 113 specifies a position where the waveform parameter is maximum, the cardiac output calculating unit 114 calculates the cardiac output based on, for example, the amplitude and the area of the microwave measured at the position (S9). The position where the waveform parameter is maximum (e.g., where the amplitude is highest, where the area is the greatest, etc.) and the cardiac output are stored in the storage unit 115. The notification unit 140 notifies the user of the cardiac output at the position where the waveform parameter is maximum from the storage unit 115 by at least one of an image, a voice, and the like (S10).

The cardiac output is calculated by selecting and using the optimum one from the microwave waveform parameters acquired in step S6 as described above. However, after identifying the position where the waveform parameter is maximum, the waveform parameter used for calculating the cardiac output may be measured again. That is, after the placing positions of the transmitting antenna and the receiving unit 126 having the maximum waveform parameter are identified in step S8, the position information of the placing positions is stored in the storage unit 115. After that, the transmitting antenna to be used is selected according to the stored position information, and the receiving unit 126 is moved at the same time. Then, the waveform parameter at the stored position may be measured again, and the cardiac output may be calculated using a value of the waveform parameter (S9).

In the present embodiment, after all the reception of the microwaves is completed (S4, S5), the waveform parameters are acquired (S6), the waveform parameters are compared (S7), the transmission and reception position of the microwave for calculating the cardiac output (S8) are determined, and then the cardiac output is calculated (S9). However, the acquisition (S6) of the waveform parameter and the comparison (S7) of the waveform parameters may be executed in parallel with the reception (S4) of the microwave, and the transmission and reception position of the microwave for calculating the cardiac output may be determined (S8) when all the reception of the microwave is completed (S5). Further, by executing the acquisition (S6) of the waveform parameters and the calculation (S9) of the cardiac output in parallel with the reception (S4) of the microwave, the calculation of the cardiac output may be completed when all the reception of the microwave is completed (S5). Furthermore, the transmission and reception position of the microwave for calculating the cardiac output may be determined using the cardiac output calculated instead of the waveform parameter or in addition to the waveform parameter.

As described above, the measuring device 100 according to embodiments of the present disclosure can measure an index related to the heart H of the living body, such as the cardiac output, and includes the transmitting unit 124, the receiving unit 126, and the detecting unit 113. The transmitting unit 124 transmits microwaves at the plurality of different locations of the living body, and measures the transmitted microwaves. The detecting unit 113 acquires waveform parameters of the microwaves measured at the plurality of locations and compares the waveform parameters. Based on the comparison result, the transmitting unit 124 and the receiving unit 126 that measure the microwave used for calculating the index among the microwaves measured at the plurality of locations are positioned.

In the measuring method, microwaves are transmitted through the plurality of different locations of the living body, and the transmitted microwaves are measured. Then, waveform parameters of microwaves measured at the plurality of locations are acquired and compared. Then, based on the comparison result of the waveform parameters, a measurement location of a microwave used for calculating an index among microwaves measured at the plurality of locations is positioned.

As described above, a waveform of a microwave received by the receiving unit is buried in noise as a distance from the heart H increases, which may affect the measurement accuracy of an index related to the heart. However, the detecting unit 113 compares the waveform parameters of the microwaves acquired at the plurality of locations and selects the waveform parameter used for calculating the cardiac output. Therefore, as described above, it is possible to measure the index related to the heart such as the cardiac output, which can change according to a position, with relatively high accuracy.

The transmitting unit 124 includes the plurality of transmitting antennas 124a to 124i. The receiving unit 126 can be disposed at a position facing the plurality of transmitting antennas 124a to 124i in a state in which the living body is interposed. With this configuration, it is possible to improve the accuracy of the index related to the heart H, such as the cardiac output, by receiving the microwaves at the plurality of different locations and comparing and selecting the waveform parameters of the plurality of acquired microwaves.

When a plurality of transmitting locations of microwaves are present as in the transmitting antennas 124a to 124i, the transmitting locations of microwaves switch between the transmission state and the non-transmission state of microwaves by a certain order. Accordingly, microwaves can be quickly transmitted at the plurality of locations, and the transmitting unit 124 and the like used for calculating the index such as the cardiac output can be efficiently positioned.

In some embodiments, the waveform parameter corresponds to the amplitude, and the detecting unit 113 positions the transmitting unit 124 and the receiving unit 126 that measure the microwave having the largest amplitude. With such a configuration, it is possible to improve the measurement accuracy of the index related to the heart such as the cardiac output. The above-described waveform parameter may be the amplitude intensity of the waveform data processed by a bandpass filter, a lowpass filter, or the like, or an A/D value may be used as the amplitude intensity of the waveform data before the filter processing.

In the transmitting unit 124 and the receiving unit 126, the table 123a of the first placing portion 123 of the moving unit 121 on which the transmitting unit 124 is mounted includes a plurality of places where the plurality of transmitting antennas 124a to 124i are placed, and includes a planar pedestal larger than the heart H in the plan view. By implementing the table 123a in this manner, microwaves can be transmitted from the plurality of different locations, and the measurement accuracy of the cardiac output and the like can be improved.

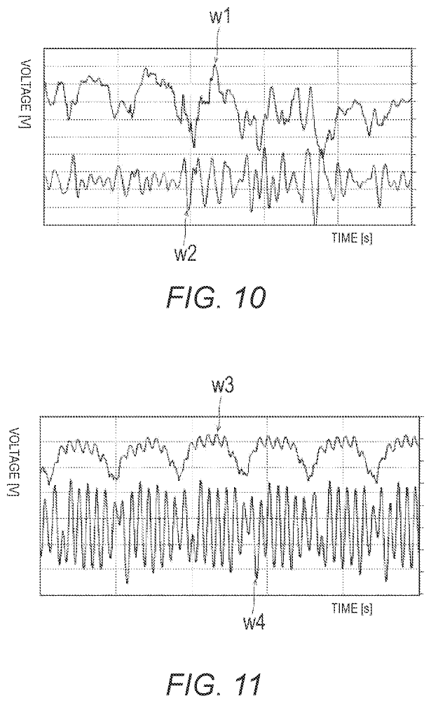

FIGS. 10 and 11 illustrate a modification and are diagrams illustrating waveforms processed by a signal processing unit. Although the waveform parameter described above may correspond to the amplitude of the waveform, alternative waveform parameters may be used. In the modification, since the waveform parameters processed by the signal processing unit 112 and compared by the detecting unit 113 are different from the above-mentioned amplitudes and the others are the same as those according to the first embodiment, the description of the common configuration will be omitted.

The detecting unit 113 compares the waveforms acquired at the plurality of locations using the autocorrelation of the waveforms of the received microwaves as the waveform parameters instead of the amplitude of the waveforms of the microwaves in the present modification, and positions the transmitting unit 124 and the receiving unit 126 that measure the microwaves used to calculate the cardiac output. The autocorrelation is a technique for evaluating a frequency at which a specific waveform periodically appears, and an autocorrelation value is a numerical value for evaluating the similarity of waveform data at a specific offset value. It can be said that, as the autocorrelation value is larger, the beat from the heart H appears periodically, and it can be evaluated that the measurement is performed at a position closer to the heart H.

In the modification, the signal processing unit 112 executes numerical analysis, filtering, and the like on the signal obtained by the reception waveform preprocessing unit 128 converting the microwave received by the receiving unit 126. Then, the autocorrelation value is calculated based on the obtained waveform. The detecting unit 113 compares the autocorrelation values calculated by the signal processing unit 112, and selects a combination of the positions of the transmitting unit 124 and the receiving unit 126 in which the microwave having the largest autocorrelation value as compared with each of the plurality of different locations is measured.

In FIGS. 10 and 11, waveforms w1 and w3 correspond to waveforms before being processed by the signal processing unit 112, and waveforms w2 and w4 correspond to waveforms processed by the signal processing unit 112. In the waveform diagram illustrated in FIG. 10, where a horizontal axis represents time, waveform w2 may not depict a pattern associated with a change in time; in other words, the waveform w2 may be relatively random over time. On the other hand, in the waveform w4 illustrated in FIG. 11, the same shape is repeated at a constant cycle as compared with that in FIG. 10, such that unnecessary noise is relatively removed. That is, from a viewpoint of the autocorrelation value, the autocorrelation value is higher in FIG. 11 than that in FIG. 10, and it can be determined that a waveform at a position close to the heart H is acquired.

As described above, in the present modification, the autocorrelation of the waveforms of the microwaves transmitted from the transmitting unit 124 and received by the receiving unit 126 is used as the waveform parameter. Then, the combination of the positions of the transmitting unit 124 and the receiving unit 126 that measure the microwave having the largest autocorrelation is determined as the positions of the transmitting unit 124 and the receiving unit 126 that measure the microwave used to calculate the cardiac output. Accordingly, it is possible to improve the measurement accuracy of the index related to the heart, such as the cardiac output.

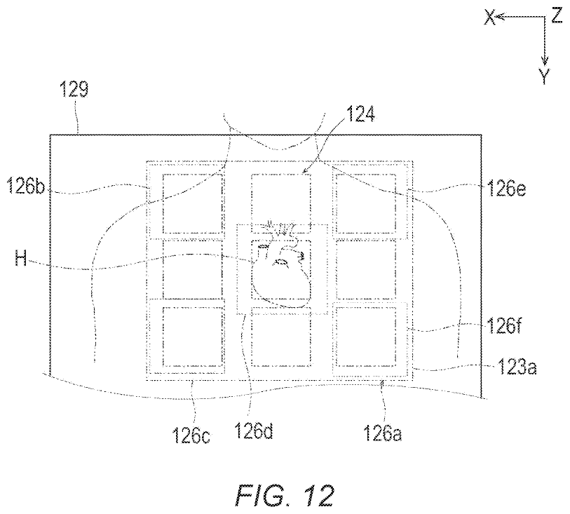

FIG. 12 is a schematic diagram illustrating an arrangement (positional relationship) between the transmitting unit 124 and a receiving unit 126a according to an alternative embodiment of the present disclosure. In some embodiments, the receiving unit 126 moves according to the positions of the microwaves transmitted from the plurality of transmitting antennas 124a to 124i. However, it is possible to adopt an alternative configuration. In the present embodiment, the second placing portion 125 on which the receiving unit 126 and the receiving antenna are placed differently, while the other components may be similar or the same as in other embodiments of the present disclosure. Therefore, the description of the common configuration is omitted. Similarly, the sizes, ratios, shapes, and hardness of the members or the number of antennas is exaggerated for convenience of description, and may be different from the actual sizes and ratios.

The transmitting unit 124 includes the plurality of transmitting locations of microwaves, such as the transmitting antennas 124a to 124i. The receiving unit 126a includes a plurality of receiving antennas as receiving locations of a plurality of microwaves, and in the present embodiment, includes five receiving antennas 126b to 126f as illustrated in FIG. 12. Similar to the first placing portion 123, a second placing portion includes a table on which the plurality of receiving antennas 126b to 126f are placed. In the present embodiment, the receiving unit 126a is not movable in the first direction X and the second direction Y, but is fixed to the table of the second placing portion. The transmitting unit 124 and the receiving unit 126a are provided in the measuring unit in the present embodiment.

As illustrated in FIG. 12, the receiving antennas 126b to 126f face a part of the positions of the transmitting antennas 124a to 124i including the transmitting unit 124 in the present embodiment. The receiving antennas 126b to 126j can receive microwave signals at various combinations of locations by being implemented in this way.

For example, in FIG. 12, since the measurement is performed by five receiving antennas for one transmitting antenna, as the relative positions of the transmitting antennas, the receiving antennas, and the heart, microwave signals at 45 different locations in total can be received.

In the transmission and reception of microwaves, switching is performed such that only a specific antenna is fed in either a plurality of transmitting antennas or a plurality of receiving antennas, and microwaves are transmitted and received only by the fed transmitting antenna and receiving antenna. Then, by this switching, the optimum microwave transmission and reception position for measurement is identified and selected by selecting the transmitting antenna and moving the receiving unit 126.

The cardiac output may be calculated by selecting the optimum one from the waveform parameters obtained in a process of identifying the optimum microwave transmission and reception position for measurement. After the transmission and reception position where the waveform parameter is maximum is identified, the waveform parameter used for calculating the cardiac output may be measured again.

The intensity of the waveform parameters may be compared in parallel while the microwave is received, or the cardiac output may be further calculated in parallel while the microwave is received.

As long as microwaves can be acquired at various locations, the arrangement of the receiving antennas is not limited to that in FIG. 12. In addition to the above, the receiving antennas may be arranged corresponding to the positions of the transmitting antennas 124a to 124i and may face the transmitting antennas in the same number as the transmitting antennas.

The number of receiving antennas may be one. In this case, the receiving antenna does not move and does not move from the initial placing location. In this case, it is desirable that the receiving antenna has a high gain by, for example, increasing the sizes of the receiving antennas in order to allow the microwaves of the transmitting antennas 124a to 124i to be received.

The sizes of the transmitting antennas 124a to 124i are not particularly limited, but in view of the volume of the heart or a left ventricle to be measured, the sizes are preferably 2 cm square or less, and more preferably 1 cm square or less.

In a case of the measuring method according to embodiments of the present disclosure, a plurality of combinations of transmitting antennas and receiving antennas that transmit and receive microwaves are designated in order in a program stored in the storage unit. Then, the receiving unit 126a receives the microwaves transmitted from the transmitting unit 124 without moving when designated by the processor 111. The remainder of the method may be carried out in accordance with embodiments of the present disclosure, and thus the description thereof is omitted.

As described above, in the present embodiment, the measuring unit includes the transmitting unit 124 capable of transmitting microwaves and the receiving unit 126a that receives microwaves. The transmitting unit 124 includes a plurality of transmitting locations implemented by the transmitting antennas 124a to 124i, and the receiving unit 126a includes a plurality of receiving locations of microwaves implemented by the receiving antennas 126b to 126f. With this configuration as well, the measurement accuracy of the index related to the heart H can be improved by comparing and selecting the waveform parameters of the microwaves acquired at the plurality of locations.

The present disclosure is not limited to the above-described embodiments, and various modifications can be made within the scope of the claims.

Although an embodiment has been described above in which the transmitting unit 124 is disposed on a front side of the human body of the patient P and the receiving unit 126 is disposed on a back side of the patient P, a specific aspect is not limited thereto as long as microwaves can be transmitted through the patient P. In addition to the above, a case in which the transmitting antenna is disposed on the back side of the human body and the receiving antenna is disposed on the front side of the human body is also included in one embodiment of the present disclosure. The transmitting antenna and the receiving antenna may be each disposed on a side surface side of the human body in a state of facing each other.

The embodiments have been described above in which one or a plurality of receiving antennas constituting the receiving unit are provided, but the present disclosure is not limited thereto. In addition to the above, a plurality of microphones or the like may be provided in a vicinity of the receiving unit 126 to serve as a heart sound detecting unit capable of detecting a heart sound as a microphone array, and the receiving antenna and the heart sound detecting unit may be moved to the periphery of the heart H.

In this case, a plurality of transmitting units 124 are provided as described herein. In the present modification, a method for transmitting and receiving microwaves from a plurality of different locations is provided as follows, instead of a program for specifying an order of transmitting the microwaves from the transmitting antennas 124a to 124i described above. That is, the receiving unit 126 and the heart sound detecting unit are moved toward the heart H from a plurality of directions corresponding to an outside of the heart H when viewed from the third direction Z in which microwaves are transmitted.

The plurality of directions can be, for example, a positive side in the first direction X, a negative side in the first direction X, a positive side in the second direction Y, and a negative side in the second direction Y. When the heart sound detecting unit detects a heart sound, a processor transmits and receives microwaves at a position of the heart sound using the transmitting unit 124 and the receiving unit 126 in the same manner as described above.

Then, the same operation as described above may be repeated in each of the plurality of directions to acquire microwave data at the plurality of different locations, and waveform parameters may be acquired and compared by the same operation as in the first embodiment to select a position where the waveform parameter is maximum. With this configuration as well, it is possible to improve the measurement accuracy of the index related to the heart H, such as the cardiac output, that can change according to the position.

In some embodiments, the transmitting unit 124 corresponds to one of the transmitting unit and the receiving unit and includes the plurality of transmitting antennas 124a to 124i. The receiving unit 126 corresponds to the other of the transmitting unit and the receiving unit, and moves according to any one of the positions of the transmitting antennas 124a to 124i that transmit microwaves. However, the present disclosure is not limited thereto. On the contrary, a case in which a plurality of receiving antennas are provided at fixed positions and the transmitting antennas are moved to any one of the positions of the plurality of receiving antennas to transmit microwaves is also included in another embodiment of the present disclosure. In some examples, the transmitting unit 124 and the receiving unit 126 may be arranged in any positioning configuration where the patient P is disposed between the transmitting unit 124 and the receiving unit 126 (e.g., while lying on the bed 129, etc.). For example, the transmitting unit 124 may be disposed in the table 125c of the second placing portion and the receiving unit 126 may be disposed in the table 123a of a first placing portion 123 of the moving unit 121. In this positioning configuration, the transmitting unit 124 may be arranged on the front side of the human body and the receiving unit may be arranged on the back side of the human body while the patient P is lying supine.

Although it has been described in the specification that the measuring device measures the cardiac output, a blood volume sent out from the heart includes not only the cardiac output but also an index such as the single cardiac output or the cardiac coefficient. Since these indexes can be converted to one another, the “index related to the heart” in the present disclosure is not limited to the cardiac output, and includes the single cardiac output, the cardiac coefficient, and other convertible indexes.

In some embodiments, an electromagnetic wave having a frequency of 0.4 GHz to 1.0 GHz is used as the microwave. The microwave may also be defined as an electromagnetic wave having a frequency of 300 MHz to 300 GHz or an electromagnetic wave having a frequency of 3 GHz to 30 GHz. As the index related to the heart such as the cardiac output, it is preferable to set a frequency at which the waveform for obtaining the cardiac output or the like is most clearly obtained, and other than the above, electromagnetic waves such as a short wave, a very short wave, and an extremely short wave may be used.

What is claimed is:

1. A measuring device configured to measure an index related to a heart of a living body, the measuring device comprising:
a measuring unit configured to transmit microwaves at a plurality of locations of the living body and measure the transmitted microwaves;
a comparing unit configured to acquire waveform parameters of the microwaves measured at the plurality of locations and compare the waveform parameters; and
a positioning unit configured to position the measuring unit that measures the microwave used to calculate the index among the microwaves measured at the plurality of locations based on a comparison result obtained by the comparing unit.

2. The measuring device of claim 1, wherein the measuring unit includes a transmitting unit configured to transmit the microwaves and a receiving unit configured to receive the microwaves, and wherein one of the transmitting unit and the receiving unit includes a plurality of transmitting locations or receiving locations of the microwaves, and wherein the other of the transmitting unit and the receiving unit is capable of being placed at a position facing the plurality of transmitting locations or the plurality of receiving locations.

3. The measuring device of claim 1, wherein the measuring unit includes a transmitting unit configured to transmit the microwaves, and a receiving unit configured to receive the microwaves,
wherein the transmitting unit includes a plurality of transmitting locations of the microwaves, and
wherein the receiving unit includes a plurality of receiving locations of the microwaves.

4. The measuring device of claim 3, wherein the plurality of transmitting locations of the microwaves disposed in the transmitting unit are configured to switch between a transmission state and a non-transmission state of the microwaves in a certain order.

5. The measuring device of claim 4, wherein the waveform parameters include at least one of an amplitude or an area of the microwaves, and
wherein the positioning unit positions the measuring unit to measure a microwave having at least one of a largest amplitude or a greatest area.

6. The measuring device of claim 4, wherein the waveform parameters include an autocorrelation of a microwave, and wherein the positioning unit positions the measuring unit to measure a microwave having the largest autocorrelation.

7. The measuring device of claim 3, wherein at least one of a portion including a plurality of the transmitting locations of the microwaves or a portion including a plurality of the receiving locations of the microwaves includes a pedestal on a surface larger than the heart in a plan view.

8. The measuring device of claim 1, wherein the index is a cardiac output, a single cardiac output, or a cardiac coefficient.

9. A measuring method for measuring an index related to a heart of a living body, the measuring method comprising:
transmitting microwaves at a plurality of different locations of the living body and measuring the transmitted microwaves;
acquiring waveform parameters of the microwaves measured at the plurality of locations and comparing the waveform parameters; and
positioning a measurement location of the microwave used to calculate the index among the microwaves measured at the plurality of locations based on a comparison result of the waveform parameters.

10. The method of claim 9, further comprising:
switching between a transmission state and a non-transmission state of the microwaves in a first order.

11. The method of claim 10, wherein the waveform parameters include at least one of an amplitude or an area of the microwaves.

12. The method of claim 11, wherein the measurement location is a location that has at least one of a largest amplitude or a greatest area of the microwaves.

13. The method of claim 11, wherein the waveform parameters include an autocorrelation of the microwave, and wherein the measurement location is a location that has the largest autocorrelation.

14. The method of claim 11, wherein the index is a cardiac output, a single cardiac output, or a cardiac coefficient.

15. A measuring device that measures an index related to a heart, the measuring device comprising:
a measuring unit configured to transmit microwaves at a plurality of different locations and measure the transmitted microwaves;
a comparing unit configured to acquire waveform parameters of the microwaves measured at the plurality of locations and compare the waveform parameters, the waveform parameters including at least one of an amplitude and an area of the microwaves; and
a positioning unit configured to position the measuring unit at a plurality of locations based on a comparison result obtained by the comparing unit.

16. The measuring device of claim 15, wherein the measuring unit includes a transmitting unit configured to transmit the microwaves and a receiving unit configured to receive the microwaves, wherein transmitting unit is positioned on a first side of a living body, and wherein receiving unit is positioned on a second side of the living body.

17. The measuring device of claim 15, wherein the positioning unit positions the measuring unit to measure a microwave having at least one of a largest amplitude or a greatest area.

18. The measuring device of claim 15, wherein the waveform parameters include an autocorrelation, and wherein the positioning unit positions the measuring unit to measure a microwave with the largest autocorrelation.

19. The measuring device of claim 16, further comprising:
a transmitting unit including a plurality of transmitting locations, wherein the plurality of transmitting locations are configured to switch on and off in a first order.

20. The measuring device of claim 15, wherein the index is a cardiac output, a single cardiac output, or a cardiac coefficient.
